(19) United States
(12) Patent Application Publication
(10) Pub. No.: US 2015/0320504 A1
(43) Pub. Date: Nov. 12, 2015

(54) MAIN BODY AND SYSTEM HAVING MULTIPLE NOZZLES FOR CLEANING A MEDICAL PRODUCT

(71) Applicant: Hysolena GmbH, Rothenburg (CH)

(72) Inventor: Lothar Saiger, Duermentingen (DE)

(21) Appl. No.: 14/791,226

(22) Filed: Jul. 2, 2015

Related U.S. Application Data
(63) Continuation of application No. PCT/EP2014/050231, filed on Jan. 8, 2014.

(30) Foreign Application Priority Data
Jan. 8, 2013 (DE) 10 2013 000 064.5

(51) Int. Cl. A61B 19/00; B08B 3/02; A61L 2/22; B08B 1/00

(52) U.S. Cl. CPC A61B 19/34; B08B 1/002; B08B 1/006; B08B 3/02; A61L 2/22

(57) ABSTRACT

A base body for cleaning or disinfecting a medical product includes a plurality of nozzles for releasing a cleaning medium. The base body is of a disc-based construction consisting of a plurality of plates arranged in succession. A cover plate can be connected to a supply line for a cleaning medium. A distributor plate is fluidically downstream from the cover plate including inlet lines. A nozzle plate is provided with the nozzles being fluidically connected to the inlet lines and open towards a face of the nozzle plate. An end cover plate is fluidically downstream from the cover, distributor and nozzle plate. The cover and nozzle plate each have a central through-clearance which is of a shape corresponding to the article's peripheral contour, where the nozzles are arranged lined up in succession along the inner periphery of the nozzle plate and being open towards the through-clearance.

### CROSS-REFERENCE TO RELATED APPLICATIONS

This continuation application claims priority to PCT/EP2014/050231 filed on Jan. 8, 2014 which has published as WO 2014/108441 A1 and also the German application number 10 2013 000 064.5 filed on Jan. 8, 2013, the entire contents of which are fully incorporated herein with these references.

### DESCRIPTION

### Field of the Invention

The invention relates to a base body comprising a plurality of nozzles and to a system comprising a base body of this type for cleaning or disinfecting an article, in particular a medical product, such as a medical or dental instrument or a dental impression. The invention further relates to a method for producing the base body.

### BACKGROUND OF THE INVENTION

Before being reused on a patient, medical products have to undergo a hygiene sequence consisting of cleaning, disinfection and if appropriate sterilization. As mechanical a preparation method as possible should be used so as to provide reproducible cleaning, disinfection and sterilization results. In practice, however, no mechanical (cleaning) system is available which is suitable for effective cleaning, disinfection and if appropriate sterilization of medical products having a rough surface and/or complex shaping, such as bone scrapers, mills or dental impressions, or of medical products comprising cavities, for example drills, ultrasound probes and ultrasound handpieces in the form of insulated handpieces or dental handpieces having a rigid sleeve. Thermolabile articles such as dental impressions are thermolabile and have to be chemically disinfected.

DE 20 2012 104279 U1 discloses a nebulizing structure comprising a plurality of nozzles for releasing a medium, which is of a disc-based construction consisting of a plurality of plates arranged in succession. The base body comprises a cover plate and a distributor plate, which is fluidically downstream from the cover plate and is provided with inlet lines for distributing the medium to the nozzles. The nozzles are arranged on a nozzle plate or nebulizing plate and are open towards the end face of the nozzle plate. An annular end cover plate serving as a drive element is positioned on the end face of the nozzle plate.

WO 2012/101642 A2 discloses an atomizer device comprising nozzles for atomizing a liquid medium, which is constructed from a plurality of plates arranged so as to lie on top of one another. A similar atomizer device is known from WO 2012/101642 A2.

The object of the present invention is to provide a base body comprising nozzles for releasing a cleaning medium or a plurality of cleaning media, in other words a cleaning head, and a system comprising a base body of this type for cleaning or disinfecting an article, in particular a medical product, such as a medical and dental instrument and a dental impression, which make possible cleaning or disinfection which is improved as a whole, even of an article which is difficult to clean, and which simultaneously is of a particularly complex construction. Further, a cost-effective and low-complexity method for producing the aforementioned base body is to be provided.

### SUMMARY OF THE INVENTION

The base body comprises a plurality of nozzles for releasing a cleaning medium, which is used in the external cleaning or external disinfection of the article, in particular medical product, to be cleaned, and according to the invention is of a disc-based construction consisting of a plurality of plates arranged in succession. The base body comprises (at one end) a cover plate, which can be connected to a supply line for the cleaning medium. The cover plate is fluidically downstream from a distributor plate, which is provided for distributing the cleaning medium to the nozzles comprising inlet lines. The distributor plate is fluidically downstream from a nozzle plate, which is provided with the nozzles. The nozzles are fluidically connected to the inlet lines of the distributor plate and formed open towards a peripheral face of the nozzle plate. The base body further comprises (at the other end) an end cover plate.

Thus, in the base body or cleaning head according to the invention, the nozzle plate is arranged between the distributor plate and the end cover plate, in such a way that cleaning medium flowing to the nozzle plate via the assigned distributor plate can be passed out of the base body via the nozzles of the nozzle plate. As a result of the construction according to the invention of the base body, the nozzles of the nozzle plate may be tightly sealed on both sides of the nozzle plate in the axial direction of the base body by the respective directly adjacently arranged plate of the base body. If the base body is only provided with one nozzle plate, it is arranged between the distributor plate, which is fluidically directly upstream from the nozzle plate, and the end cover plate. The plates may in particular lie tightly (directly) against one another in the region of the contact faces thereof. Between the individual plates of the base body, preferably annular sealing elements or seals may also be arranged. The sealing elements/seals preferably consist of an elastomeric, in other words rubbery-elastic or viscoplastic material. In particular PTFE has been found expedient as a viscoplastic material.

According to the invention, the inlet lines to the nozzles are formed in the distributor plate of the base body. According to the invention, the inlet lines may be configured in such a way that there is a separate inlet line for each nozzle or in such a way that each inlet line interconnects a plurality of nozzles. The inlet lines may at least in portions be formed as groove-like depressions in the distributor plate. Alternatively, the inlet lines may also be formed as through-clearances in the distributor plate.

In the present application, the term “cleaning medium” refers not only to cleaning media such as water, cleaning agents or the like, but also to disinfectant solutions or compressed air or other liquids or gases, for example for gas sterilization using ozone or other gases.

Thus, according to the invention, the base body is formed from a plate stack and can thus be manufactured particularly compact and in a cost-effective manner. For the purpose of low susceptibility to wear, the nozzle plate may in particular be manufactured from a high-quality, abrasion-resistant and thermally resistant material, such as steel or a steel alloy. The nozzles may be worked out of the material of the nozzle plate in a simple manner. This can be done in particular by a laser-cutting method, but also by grinding, milling, punching or drilling. The base body may further be adapted individually to different cleaning tasks, in other words for cleaning a specific article or medical product, in a simple manner. According to the invention, the base body comprises a preferably central through-clearance which extends through the base body in the longitudinal direction of the base body, in other words transversely to the individual plates of the base body. As a result, the base body can be moved in translation along the article to be cleaned in such a way as to encompass the article to be cleaned, so as to treat or clean/disinfect said article completely from the outside. In this case, the nozzles of the base body are oriented or open towards the through-clearance of the base body.

Thus, according to the invention, the nozzles are arranged in sequence at the edge along the inner periphery of the nozzle plate. The base body is matched to the shape/contour of the article to be cleaned or has a shape and thus peripheral contour imitating the shape/contour of the article to be cleaned. As a result, the nozzles are arranged, in an extremely constructionally simple manner, along the peripheral contour of the nozzle plate and thus of the base body, which corresponds to the outer contour of the article to be cleaned. The nozzles of the nozzle plate may further be individually tuned to the article (medical product) to be cleaned in terms of the spatial arrangement thereof on the base body or the respective spray characteristics thereof in an extremely simple manner. As a result, the cleaning medium can be applied to the article to be cleaned in a targeted manner during a cleaning process. The nozzles may have the same spray characteristics or differ at least in part in the respective spray characteristics thereof. According to the invention, the nozzles may in particular be tuned in a targeted manner, in terms of the opening cross section, the shape of the opening, the spray angle (=opening angle; spray vs. jet), the primary flow direction (angle of approach of the nozzle) relative to the longitudinal axis of the base body or of the article to be cleaned, and/or the cross section and/or the length of the respective inlet lines or supply lines for the cleaning medium in the distributor plate, to the article to be cleaned, in other words to the surface contour (shape) thereof or surface portions thereof which are critical in terms of cleaning. As a result, the nozzles can carry out different cleaning tasks in each case. Overall, a defined cleaning result can thus be provided particularly efficiently and reproducibly whilst conserving resources.

In a preferred development of the invention, the base body comprises a plurality of nozzle plates, to each of which a distributor plate is assigned. Between every two nozzle plates, a distributor plate is arranged which is assigned to the nozzle plate fluidically directly downstream. As a result, the base body can be provided in a simple manner with a plurality of nozzles which are arranged spaced apart in the longitudinal direction of the base body. In this way, the cleaning or disinfection of the article can again be further improved and accelerated. It will be appreciated that in this case the distributor and nozzle plates are provided at least in part with through-clearances through which the cleaning medium can respectively be supplied to the distributor plate fluidically downstream.

For supplying different cleaning media to the nozzle plates, in the simplest possible construction the base body comprises a separator plate which is arranged between a first and a second nozzle plate. The separator plate is thus fluidically upstream from the distributor plate of the second nozzle plate. It will be appreciated that in this case the cover plate is provided with separate passages for the cleaning media, and the distributor plate of the first nozzle plate, the first nozzle plate, and the separator plate are each provided with a passage for the cleaning medium intended for the second nozzle plate.

According to the invention, the cover plate and/or the end cover plate may be provided with nozzles. The cleaning medium is supplied to the nozzles of the cover plate via the distributor plate, which is arranged directly fluidically downstream from the cover plate. With a view to a compact and simple construction of the base body, the cleaning medium is advantageously supplied to the nozzles of the end cover plate via the nozzle plate of the base body which is directly fluidically upstream from the end cover plate.

The plates of the base body may in particular be screwed or hooked together, or welded, soldered or glued together. In the first case, the plates may advantageously each be provided with at least two through-holes for connection means, the through-holes in the plates being mutually flush in the axial direction. The connection means used extend through the through-holes of the individual plates so as to interconnect or clamp together the plates. In the last three cases, additional sealing elements between the individual plates may be superfluous.

For further improved cleaning of the article or medical product, one or more additional cleaning elements, for example brushes or cleaning cloths, may be attached to the base body. According to the invention, the base body may comprise a turbine rotor, by means of which the additional cleaning elements are movable about an axis of rotation relative to the base body. In the simplest possible construction, the turbine rotor can be driven by the cleaning medium supplied to the base body. A portion of the volume flow of the cleaning medium supplied to the base body is supplied to the turbine rotor for this purpose.

The cover plate of the base body is preferably configured in such a way that a plunger can be mounted thereon, by means of which the base body can be moved back and forth and by means of which the cleaning medium can be supplied to the base body. As a result, the base body can also be used in a (cleaning) system, in other words in a cleaning machine. By means of the plunger, the base body can be moved relative to the articles to be cleaned or processed during cleaning or disinfection operation or processing operation.

According to the invention, to heat the cleaning medium or cleaning media the base body may comprise a heating element. The heating element may either be formed by one or more plates of the base body itself or else be formed as a component separate from the plates of the base body. In the first case, the plate(s) is/are formed as a resistance heating member.

An electrical connection line of the aforementioned heating member may be supplied to the base body for example via the aforementioned plunger. Alternatively or in addition, the plunger may be provided with a heater for heating the cleaning medium/cleaning media.

According to the invention, the base body may further be formed as a light emission unit, to emit light of a defined wavelength or a defined wavelength spectrum onto an article to be treated, in particular to be cleaned or disinfected or even sterilized. The light can preferably be coupled into the base body from the outside, for example via an optical fiber. The base body may consist at least in part of a light-conducting material. Further, the base body may be provided with a deflection means, for example a mirror, by means of which the light beam can be deflected in a targeted manner onto the article to be treated. In particular UV light may be used for disinfecting the article.

The method for producing an aforementioned base body comprises the following method steps:

Initially, the nozzles are formed in the nozzle plate, in such a way that the nozzles are arranged lined up in succession along the inner periphery of the nozzle plate and are open towards the through-clearance. In a further step, the inlet lines are formed in the distributor plate.

The nozzles and/or inlet lines may be cut out of the respective plate using a laser or else be drilled, milled or ground.

In a further step, the cover plate and the end cover plate are provided.

In a further step, the cover plate, the distributor plate, the nozzle plate and the end cover plate are laid or stacked on one another and interconnected to form the base body. This may be done by welding, gluing, soldering or screwing the aforementioned plates.

The method according to the invention makes possible particularly simple and cost-effective production of base body comprising a plurality of nozzles, at least some of which may have a shared supply line for the cleaning medium/media or which may have a separate supply line in each case.

According to the invention, sealing elements, which seal the plates against one another and fluid-tightly against one another in the radial direction, may be arranged between the individual plates. The sealing elements may in particular be positioned loosely between the plates and be arranged held in a clamped fit between the plates as a result of the subsequent interconnection thereof.

For a particularly secure tight fit of the sealing elements, the plates may be provided on the mutually facing side faces thereof with a seal-receiving structure, in particular an annular groove or the like, in which the sealing elements are laid.

The system according to the invention for cleaning an article, in particular a medical product, comprises in a manner known per se a preparation chamber in which the article to be cleaned or the medical product is arranged. The system is provided with at least one aforementioned base body, the base body being mounted on a plunger or on another linear drive, in such a way that the base body can be moved back and forth, relative to the article, in the preparation chamber for cleaning the article or medical product, by means of the plunger or linear drive.

The base body is thus connected to a plunger in or on which there is preferably a piping, through which the cleaning medium or media can be supplied to the base body and thus to the nozzles. Various cleaning media can thus be transported to the base body through one inlet line or else through different inlet lines.

The plunger may be part of a piston which moves the plunger in such a way that the base body comprising the nozzles is guided along on the article to be cleaned and thus cleans and/or chemically disinfects the entire length of the article. The piston is preferably electrically or pneumatically driven. A cylinder may also be used instead of a piston.

For simplified handling of the system, the preparation chamber of the (cleaning) system may comprise a container which is permeable to the cleaning medium and in which the article or articles/medical products to be cleaned can be laid. In the simplest possible construction, the container may be formed as a lattice box. The container may preferably be removable from the preparation chamber. As a result, the preparation chamber can be loaded with the article to be cleaned in a simplified manner and said article can be removed from the preparation chamber in a simplified manner after a completed cleaning process.

The container preferably comprises mountings for the article or medical product to be cleaned, which are individually tuned to the article or medical product to be cleaned. In this way, it can be ensured that the article to be cleaned is arranged in a (predetermined) position, orientated for optimal cleaning of the articles, relative to the base body, in other words to the cleaning head.

According to the invention, the preparation chamber may comprise an adapter for connecting an article (medical product) formed as a hollow instrument. As a result, the cleaning medium can also be passed through the interior of the article/medical product to be cleaned.

The system preferably comprises a plurality of the aforementioned base bodies comprising nozzles. As a result, a plurality of and/or of different medical products or articles may be cleaned simultaneously or with a time offset. This is advantageous for the efficiency and flexibility of use of the system. The plungers of the base body are thus preferably spaced apart and arranged extending mutually parallel, in such a way that the base bodies can be reliably prevented from obstructing each other's movement within the preparation chamber. Further, in this way the individual base bodies can reliably be prevented from impairing each other's cleaning or disinfection capacities, and undesirable cleaning shadows are prevented. The base bodies preferably have a shared drive.

At the connection side, the system preferably comprises a valve block and a distributor block for distributing the cleaning medium or cleaning media on the base body and/or the adapter.

In a preferred development of the invention, components for cooling and/or an internal heater and/or an external heater and/or a metering unit for processing agents and/or components for monitoring the process is/are arranged in the distributor block.

### BRIEF DESCRIPTION OF THE DRAWINGS

In the following, the invention is described in greater detail by way of an embodiment illustrated in the drawings, in which:

FIG. 1 is a side view of a system according to the invention for externally cleaning or externally disinfecting an article, in this case a medical product, comprising a receiving chamber for the article to be cleaned and comprising a base body, arranged movably in the receiving chamber and comprising nozzles for releasing a cleaning medium, the base body being of a disc-based construction consisting of a plurality of individual plates stacked on one another, and the nozzles being formed at an edge portion in an individual plate of the base body formed as a nozzle plate;

FIG. 2 is a plan view of the cover plate of the base body of FIG. 1 comprising a passage for the cleaning medium;

FIG. 3 is a plan view of the nozzle plate of the base body of FIG. 1 comprising the nozzles arranged on the internal peripheral face;

FIG. 4 is a plan view of the distributor plate of the base body of FIG. 1 comprising inlet lines, formed in the distributor plate, for the nozzles of the nozzle plate shown in FIG. 3;

FIG. 5 is a side view of an alternative embodiment of the base body of FIG. 1, in which the base body is provided with additional cleaning elements;

FIG. 6 shows an alternative embodiment of the base body of FIG. 1 comprising a plurality of nozzle plate and distributor plate pairs;

FIG. 7 is a side view of an alternative embodiment of the base body of FIG. 1 comprising a plurality of nozzle plates to which different cleaning media can be supplied; and

FIG. 8 is a block diagram comprising individual method steps of the method according to the invention for producing the base body shown in FIG. 1.

### DETAILED DESCRIPTION OF THE PREFERRED EMBODIMENTS

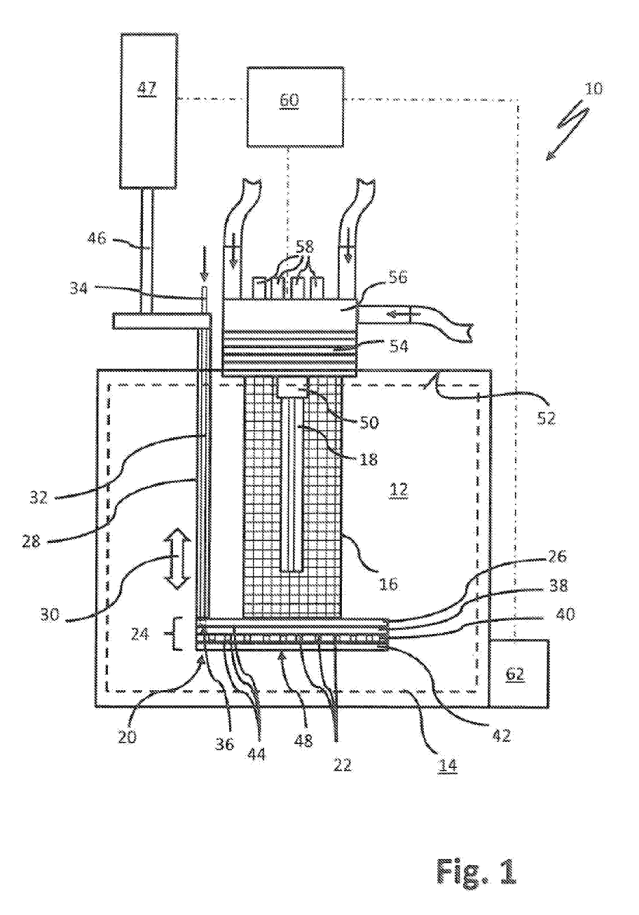

FIG. 1 is a schematic drawing of a system 10 for cleaning an article, in particular a medical product, such as a medical/dental instrument. The system 10 comprises a preparation chamber 12 comprising a cover 14 which can be opened. In the preparation chamber 12, a container 16 formed as a lattice cage is arranged in which an article 18 to be cleaned, in this case a dental instrument, is arranged. The container 16 is permeable to a cleaning medium in such a way that the article 18 can be cleaned through said container from the outside.

In another embodiment, the article 18 to be cleaned is laid in a special mounting in the container 16. In this way, particularly intensive cleaning of the article 18 can be provided, without it being able to move out of position under the pressure of the cleaning medium.

A base body 20, in other words a cleaning head, which comprises a plurality of nozzles 22 for targeted release of a cleaning medium onto the article 18, is used for external cleaning of the article 18. In the embodiment shown in FIG. 1, the base body 20 is annular in form and is of a disc-based construction consisting of a plurality of plates. In other words, the base body 20 comprises a plate stack 24. A terminating cover plate 26 of the base body 20 is used to fix the base body 20 to a plunger 28, which can be moved back and forth in the arrow direction 30. The plunger extends outwards through the wall of the preparation chamber 12.

The plunger 28 comprises in the interior thereof a line 32 via which the cleaning medium can be supplied to the base body 20. A supply line 34, in this case a hose, for the cleaning medium used in the cleaning process is connected to the line 32 on the inlet side.

The terminating cover plate 26 comprises a passage 36 for the cleaning medium. A distributor plate 38, which is used to distribute the cleaning medium to the nozzles 22 of a nozzle plate 40, is adjacent to the terminating cover plate 26. An end cover plate 42 is adjacent to the nozzle plate on the side of the nozzle plate 40 remote from the terminating cover plate 26.

The shape of the nozzles 22, the thickness of the plates, the cross section of the inlet line for the nozzles 22 and the number of nozzles 22 influence the pressure at which the cleaning medium impacts on the article 18 to be cleaned and thus the cleaning power. The construction of the nozzles 22 determines whether the cleaning medium exits the nozzle as a jet or as a spray.

The aforementioned plates of the base body 20 are screwed together (not shown in greater detail), but may also be hooked, welded, soldered or glued together or otherwise tightly interconnected. As shown in FIG. 1, a sealing element 44 made of an elastomeric material may be arranged between the individual plates in each case so as to seal the individual plates of the base body 20 fluid-tightly against one another.

The plunger is adjustably coupled in movement with a further plunger 46 arranged outside the preparation chamber. The further piston rod is guided in a pneumatic cylinder 47. In an alternative embodiment (not shown in greater detail in the drawings), the plunger 28 may also be electrically, hydraulically or magnetically drivable.

In the present example, the base body 20 comprises a (basic) shape tuned to the medical product 18 to be cleaned and having a through-clearance 48 which is larger than the container 16. As a result, when the piston rods 28, 46 are actuated, the base body 20 can be moved relative to the article to be cleaned over the container 16 and the article to be cleaned arranged therein whilst encompassing the container 16. When the base body moves relative to the article 18 to be cleaned, the article 18 thus extends through the through-clearance 48 in the base body. The through-clearance 48 in the base body 20 is formed by individual through-clearances in the plates of the base body 20. The nozzles 22 of the base body 20 are arranged on the inner peripheral face of the base body 20 and open towards the through-clearance 48 in the base body 20.

The present system is further configured for internally cleaning hollow instruments, such as ultrasound handpieces or dental turbine handpieces or the like.

For this purpose, the preparation chamber 12 comprises an adapter 50 for connecting the article 18 to be internally cleaned or disinfected. The adapter 50 is arranged on an internal wall 52 of the preparation chamber 12 in the present case, but may also in principle be arranged on the base face of the preparation chamber 12. For dental transfer instruments having a fixed hose, for example the inlet line, through which water is pushed for example to the ultrasound probe, is connected to the adapter 50. In the present case, the article 18 formed as a hollow instrument is connected to the adapter 650.

As can further be seen from FIG. 1, the system 10 comprises a distributor block 54 comprising a valve block 56, by means of which one or more cleaning media can be supplied, simultaneously or with a time offset, to the base body 20 comprising the nozzles 22 or the adapter 50 for internally cleaning the article to be cleaned. As a result of one or more valves 58 being opened, the cleaning medium flows through the valve block 56 into the distributor block 54, which subsequently passes the cleaning medium in a targeted manner to the base body 20 for externally cleaning the article 18 or to the adapter 50 for internally cleaning the article 18 formed as a hollow body. Water, cleaning agent, disinfectant, pressurized air or gases can thus be pressed through the inlet line or the article 18 and clean and disinfect them.

During thermal disinfection or sterilization of the article 18 inside the preparation chamber 12, the residual water which remains in the inlet line after the cleaning can evaporate. Further, water can be introduced into the hot inlet line, and subsequently evaporates instantaneously. The resulting water vapor can thus be used for heat cleaning. This method also makes it possible to prepare endoscopes.

A control unit 60 is used to control all of the operating or process parameters of the system. A display and operating unit 62 of the system is used as a user interface, and is connected to the control unit 60.

It will be appreciated that the system 10 may also comprise a plurality of the aforementioned base bodies 20. The base bodies 20 may differ from one another in shaping and construction for treating differently shaped articles 18.

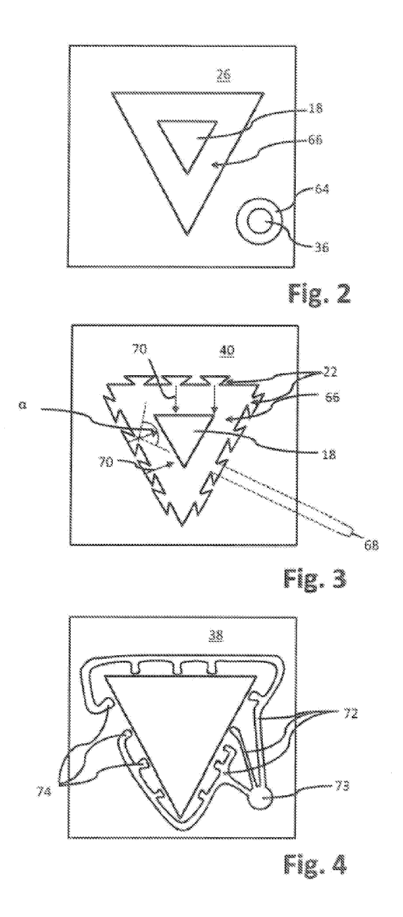

FIG. 2 is a plan view of the terminating cover plate 26 of the base body 20 shown in FIG. 1, comprising the passage 36 for the cleaning agent flowing in via the plunger. The terminating cover plate 26 comprises a fixing flange 64 for the piston rod (FIG. 1). A central through-clearance in the cover plate 26 is denoted as 66. The through-clearance 66 in the cover plate 26 is of a shape corresponding to the peripheral contour (shape) of the article 18 to be cleaned.

FIG. 3 shows an example configuration of a further individual plate of the base body 20 shown in FIG. 1, in this case the nozzle plate 40. The nozzle plate 40 comprises a central through-clearance 66, which corresponds to the central through-clearance of the cover plate shown in FIG. 2. The nozzles 22 are arranged lined up in succession along the internal circumference of the nozzle plate 40 and are open towards the through-clearance 66 of the nozzle plate 40.

In the embodiment shown, the nozzles 22 have a uniform shaping having a uniform nozzle angle α. The nozzles 22 have been cut, in other words cut free (worked), out of the nozzle plate using a laser.

The possible flow rate and the pressure of the cleaning medium are influenced by the opening cross section 68 of the nozzles. A respective primary flow axis 70 of the nozzles 22 is denoted as 68. The primary flow axis 70 of the nozzles corresponds to a respective angle of approach (orientation) of the nozzles 22 with respect to the longitudinal axis of the base body.

The position of the nozzles 22 on the nozzles plate 40 and the respective further nozzle parameters thereof, in other words the opening cross section 68 and primary flow direction 68 thereof and the inlet lines or supply lines (supply ducts) thereof for the cleaning medium in the distributor plate are orientated onto the article to be cleaned in a targeted manner, in other words onto the surface contour thereof and surface portions thereof which are critical in terms of cleaning.

FIG. 4 is a free plan view of the distributor plate 38 of the base body 20 shown in FIG. 1. The inlet lines 72 for the nozzles of the nozzle plate 40 shown in FIG. 2 can clearly be seen.

The inlet lines 72 may be formed as depressions or as through-clearances in the distributor plate 38. The inlet lines are all fluidically connected via a through-hole 73 to the passage in the terminating cover plate (FIG. 2). The inlet lines 72 may be lasered, drilled, milled or ground out of the nozzle plate, which is for example made of stainless steel.

Of course, other plate materials are also conceivable. The material of the distributor plate 38 depends, among other things, on the (cleaning) medium/media which are passed through the distributor plate 38.

The ducts or inlet lines 72 of a plurality of nozzles may be combined. As is shown in FIG. 4, an inlet line 72 of an individual nozzle may be assigned individually. The bump-like swells 74 in the supply lines provide the fluidic connection of the supply lines or inlet lines 72 for the individual nozzles of the nozzle plate shown in FIG. 2. These bump-like swells 74 are formed as through-holes in the distributor plate 38, and open into the cavity (not shown), fluidically upstream from the nozzle 22, of the nozzle plate.

The respective flow rate and the pressure of the cleaning medium at the individual nozzles depends, among other things, on the cross section of the supply line, the length of the supply line and the cleaning nozzle itself. Particular shapes in the end or outlet region of the nozzles 22 may lead to desired eddies in the cleaning medium and/or to spray formation.

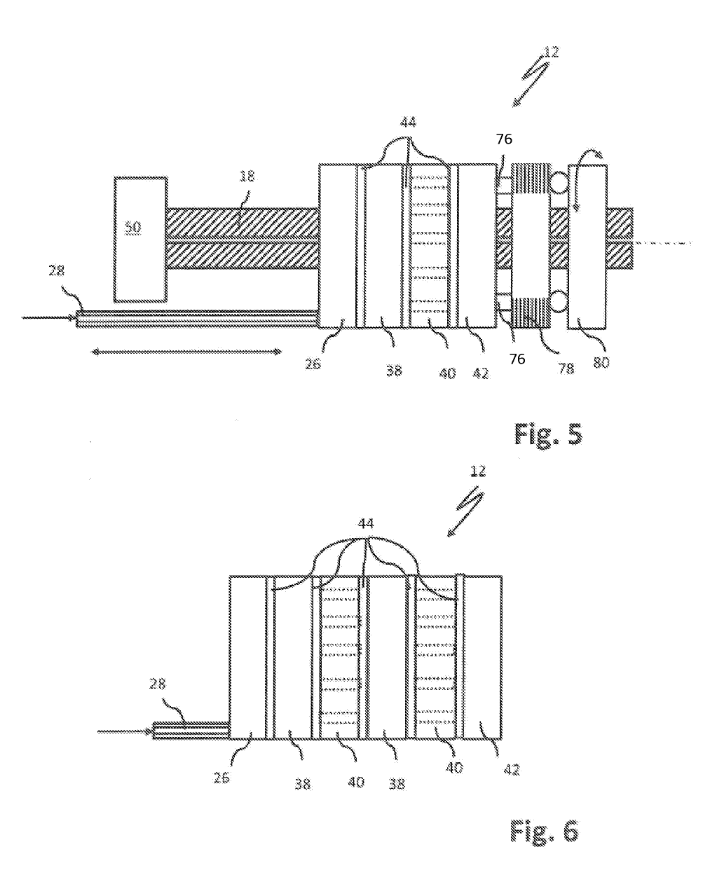

FIG. 5 shows a further embodiment of the base body 20 comprising a terminating cover plate 26, a distributor plate 38, a nozzle plate 40 and a terminating cover plate 42.

The sealing elements 44 or seals between the individual plates can clearly be seen. The plunger 28 is fixed to the terminating cover plate 26.

The base body differs from the base body described in connection with FIG. 1 in that it comprises a fixing option 76, in other words fixing means, for a first (additional) cleaning element 78. The cleaning element 78 may be in the form of brushes, as shown by way of example in FIG. 5, or also in the form of cleaning cloths (not shown)

Via a further fixture, a (further) cleaning element formed as a turbine rotor 80 may be attached to the cleaning element 78, and mounted movably thereon relative to the base body 20. For example, the movable mounting of the on the base body 20 may be provided by a ball bearing. It will be appreciated that the cleaning unit 78 may also be attached or mounted movably on the base body 20 and optionally be in the form of a turbine rotor.

In an embodiment of the base body 20 not shown in greater detail in the drawings, it may comprise an attachment configured in such a way that the instrument to be cleaned can thus be rotated by moving the base body 20.

FIG. 6 shows a further base body which basically differs from the base body shown in FIG. 1 in that it comprises two distributor plate/nozzle plate units arranged in pairs, which are connected in series.

FIG. 7 shows the basic construction of a base body 20 comprising nozzles, in which base body two different cleaning media can be used.

The base body 20 comprises two distributor and nozzle plates 38, 40 arranged in pairs in succession and separated from one another by a single separator plate 82. The base body 20 is delimited by the cover plate 26 at one end and by the end cover plate 42 at the other end.

The base body 20 is fluidically connected to two separate supply lines 32 for the two cleaning media. The supply line 32 shown on the left in FIG. 7 is fluidically connected via a first passage (not shown) of the cover plate 26 to the (first) distributor plate 38 upper in the drawing of the (first) nozzle plate upper in the drawing, and via the supply lines (not shown) of this distributor plate 38 to the nozzles (not shown) of the first nozzle plate 40.

The second supply line shown on the right in FIG. 7 is fluidically connected via a second passage (not shown) in the cover plate and through-holes in the distributor plates 38 and the first nozzle plate 40 and the separator plate 82 to the nozzles of the (second) nozzle plate lower in the drawing.

The nozzles of the two nozzle plates 40 are arranged on the base body 20 in such a way that the cleaning media of the two nozzle plates 40 mix together at least in part after leaving the nozzles. The mixing ratio is influenced by the selected flow rate of the cleaning medium through the respective nozzles and the shared coverage of the respective media jets. To form a media spray, one of the two cleaning media may be gaseous, for example compressed air.

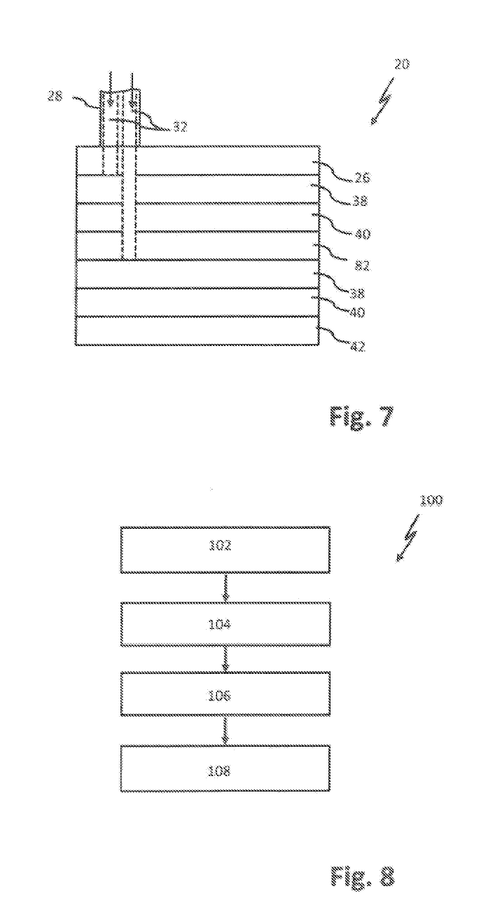

FIG. 8 is a block diagram comprising individual method steps of the method 100 according to the invention for producing the base body described in connection with FIGS. 1 to 3.

In step 102, the nozzles 22 are formed in the nozzle plate 40. In other words, the nozzles 22 are formed in a stainless steel plate or another nozzle plate blank to form the nozzle plate.

In a further step 104, the inlet lines are formed in the distributor plate. In other words, the inlet lines are formed in a stainless steel plate or another distributor plate blank to form the distributor plate.

The nozzles 22 and/or the inlet lines 72 may be cut out of the respective plate using a laser in each case or may be drilled, milled or ground out.

In a further step 106, the cover plate 26 and the end cover plate 42 are provided.

In a final step 108, the cover plate 26, the distributor plate 38, the nozzle plate 40 and the end cover plate 42 are laid or stacked on one another and interconnected to form the base body 20.

The shaping of the individual plates is selected in such a way that the peripheral face (=surface) of the base body to be formed which faces the article to be cleaned during operation is adapted to the (basic) shape or basic contour of the article 18 (FIG. 1) to be cleaned. In other words, the plates, in particular the nozzle plate, comprise at least in portions a peripheral contour tuned to, in particular corresponding to, the basic shape or basic contour of the article to be cleaned. The arrangement of the nozzles along the peripheral contour of the nozzle plate and thus of the base body and the spray properties of the individual nozzles are individually tuned to the basic shape of the article to be cleaned.

In method step 108, sealing elements 44 may be arranged between the individual plates to prevent undesired escape of the cleaning medium, which flows into the base body during operation, outside the nozzles 22 provided therefor.

By the method described, the base body 20 can be produced completely individually for the respective cleaning or disinfection task, in other words in a manner specific to the article to be cleaned, with nozzles, including the inlet lines and connections thereof.

What is claimed is:

1. A base body for cleaning or disinfecting an article including a medical product, a medical or dental instrument or a dental impression, the base body including a plurality of nozzles for releasing a cleaning medium, the base body being of a disc-based construction comprised of a plurality of plates arranged in succession, the plurality of plates of the base body comprising:
a cover plate, which can be connected to a supply line for a cleaning medium;
a distributor plate, which is fluidically downstream from the cover plate and which is provided with inlet lines for distributing the cleaning medium to the plurality of nozzles;
a nozzle plate, which is provided with the plurality of nozzles, the plurality of nozzles being fluidically connected to the inlet lines of the distributor plate and open towards a face of the nozzle plate;
an end cover plate, which is fluidically downstream from the cover plate, the distributor plate and the nozzle plate;
wherein the cover plate and the nozzle plate each comprise a central through-clearance which is of a shape corresponding to the peripheral contour of the article to be cleaned; and
wherein the plurality of nozzles are arranged lined up in succession along the inner periphery of the nozzle plate and are open towards the central through-clearance.

2. The base body according to claim 1, wherein at least some of the nozzles are arranged spaced apart differently on the nozzle plate.

3. The base body according to claim 1, including annular sealing elements arranged between the individual plates of the plurality of plates.

4. The base body according to claim 2, including annular sealing elements arranged between the individual plates of the plurality of plates.

5. The base body according to claim 1, wherein the plurality of plates of the base body are screwed, welded, soldered or hooked together.

6. The base body according to claim 1, including an additional cleaning element comprising a brush or a cleaning cloth is arranged on the base body.

7. A system for cleaning or disinfecting an article including a medical product, the system comprising:
a preparation chamber, in which the article to be cleaned is arranged; and
a base body comprising a plurality of nozzles for releasing a cleaning medium, wherein the base body comprises:
a cover plate, which can be connected to a supply line for a cleaning medium;
a distributor plate, which is fluidically downstream from the cover plate and which is provided with inlet lines for distributing the cleaning medium to the plurality of nozzles;
a nozzle plate, which is provided with the plurality of nozzles, the plurality of nozzles being fluidically connected to the inlet lines of the distributor plate and open towards a face of the nozzle plate; and
an end cover plate, which is fluidically downstream from the cover plate, the distributor plate and the nozzle plate;
wherein the cover plate and the nozzle plate each comprise a central through-clearance which is of a shape corresponding to the peripheral contour of the article to be cleaned;
wherein the plurality of nozzles are arranged lined up in succession along the inner periphery of the nozzle plate and are open towards the central through-clearance;
wherein the base body is adapted in shape to the shape of the article to be cleaned; and
wherein the base body is able to be guided along in the preparation chamber on the article to be cleaned.

8. The system according to claim 7, wherein the plurality of nozzles are respectively specifically tuned to the article to be cleaned in terms of a spray characteristic thereof.

9. The system according to claim 8, wherein the spray characteristic comprises an opening cross section, a spray angle and/or a primary flow direction thereof.

10. The system according to claim 7, wherein the base body is mounted on a plunger by means of which the base body can be guided along on the article to be cleaned.

11. The system according to claim 10, wherein the plunger comprises a line via which the cleaning medium can be supplied to the base body.

12. The system according to claim 10, wherein the plunger can be driven pneumatically or electrically.

13. The system according to claim 7, wherein the preparation chamber comprises a container which is permeable to the cleaning medium and in which the article to be cleaned is laid.

14. The system according to claim 13, wherein the preparation chamber comprises an adapter for connecting an article in the form of a hollow instrument, via which adapter the cleaning medium can be supplied to the article for internally cleaning the article.

15. The system according to claim 7, wherein the base body comprises two or more base bodies which are both arranged movably in the preparation chamber with a shared drive.

16. A method for producing a base body according to claim 1, the method comprising the steps of:
forming the nozzles in the nozzle plate, in such a way that the nozzles are arranged lined up in succession along the inner periphery of the nozzle plate and are open towards the through-clearance;
forming the inlet lines in the distributor plate;
providing the cover plate and the end cover plate; and
stacking on one another and interconnecting the cover plate, the distributor plate, the nozzle plate and the end cover plate to form the base body.

17. The method according to claim 16, wherein the plurality of nozzles in the nozzle plate and/or the inlet lines in the distributor plate are lasered out.

18. The method according to claim 16, wherein the plurality of nozzles in the nozzle plate and/or the inlet lines in the distributor plate are drilled, milled, punched or ground out of the nozzle plate and distributor plate, respectively.

19. The method according to claim 16, including sealing elements arranged between the individual plates of the plurality of plates.
